(19) United States
(12) Patent Application Publication
(10) Pub. No.: US 2007/0014758 A1
(43) Pub. Date: Jan. 18, 2007

(54) Packaging materials for ophthalmic lenses

(76) Inventor: Eoin Roche, Waterford (IE)

Correspondence Address: Bausch & Lomb Incorporated, One Bausch & Lomb Place, Rochester, NY, 14604-2701, US

(21) Appl. No.: 11/183,413

(22) Filed: Jul. 18, 2005

(51) Int. Cl. A61K 31/74

(52) U.S. Cl. 424/78.31

(57) ABSTRACT

Packaging material for ophthalmic lenses, such as contact lenses, includes graphics with at least one of a right-side facial profile or a left-side facial profile. The graphics permit designating the lens included in the packaging for right or left eye placement.

### BACKGROUND OF THE INVENTION

This invention provides packaging materials for ophthalmic lenses. The invention provides methods of identifying or distinguishing an ophthalmic lens for placement in or on the right or left eye.

Ophthalmic lenses, such as contact lenses and intraocular lenses, are usually prescribed in order to correct a patient's vision. In the case of contact lenses, the lens is placed on the patient's cornea, and in the case of intraocular lenses, the lens is implanted in the patient's eye to replace the natural lens.

Contact lenses are frequently delivered to patients in a carton containing multiple contact lenses, for example, several blister packages, each containing an individual contact lens, may be contained in a secondary carton. It is not uncommon for an individual patient to obtain different prescriptions for the right and left eyes. For example, a patient may need a contact lens with a correction of −3.00 diopter for the right eye, and a correction of −2.00 diopter for the left eye. Accordingly, a contact lens wearer, or the prescribing practitioner, may designate on the blister package and/or on the carton the eye for which the lens is to be worn.

FIG. 1 illustrates a known carton 2 that contains contact lens blister packages and includes graphics that facilitate designating the prescription as for either the right eye or the left eye. Specifically, the patient or the practitioner can place a mark in the box adjacent R (right) 3 or in the box adjacent L (left) 4. However, the R and L abbreviations for right and left do not apply to all non-English languages. Thus, for packages intended for distribution in multiple markets with different language requirements, the L and R designations, alone, may not be sufficient for all markets. Alternately, one could list various individual language equivalents of right and left (as in FIG. 1), but this approach takes up space on the package, and may still not encompass languages for all countries of distribution.

### SUMMARY OF THE INVENTION

This invention provides packaging material for ophthalmic lenses including graphics comprising at least one of a right-side facial profile or a left-side facial profile. The graphics may include both the right-side and left-side facial profiles spatially separated from one another on the packaging material, or only one of the right-side and left-side facial profiles. As examples, the right-side facial profile may be a full or partial silhouette of the right side of a human head, or the left-side facial profile may be a full or partial silhouette of the left side of a human head.

The graphics may be included on: primary packaging for lenses, such on the cover sheet of a lens blister package; a secondary carton enclosing lens primary packages; and/or an insert enclosed in a carton containing an ophthalmic lens.

The invention further provides a method for identifying an ophthalmic lens for placement in or on the right or left eye, comprising, designating the right or left eye placement on packaging material for the lens with, respectively, the right-side facial profile or the left-side facial profile. Additionally, invention provides a method for distinguishing placement of an ophthalmic lens for the right eye or the left eye, comprising, including on packaging material for the lens a right-side facial profile and a left-side facial profile that are spatially separated from one another.

The invention provides a simple identification and designation of right or left placement of a lens, in that the non-textual right- and left-side facial profiles fully convey right and left eye placement, respectively. The packaging materials may be distributed to multiple geographic areas, including non-English speaking countries and regions not using the Roman alphabet, without the need for multiple language textual descriptions of right and left eye placement.

### BRIEF DESCRIPTION OF THE DRAWINGS

FIG. 1 is a perspective view of a prior art contact lens secondary carton, showing the graphics relating to right and left designation.

FIG. 2 is a perspective view of a representative blister package containing a contact lens.

FIG. 3 is a perspective view of the blister packages of FIG. 2 contained in a secondary carton.

FIG. 4 is a perspective view of another secondary carton.

FIG. 5 is a perspective view of a contact lens blister package.

FIG. 6 is a plan view of a card for insertion in an ophthalmic lens package.

FIG. 7 is a plan view of another card for insertion in an ophthalmic lens package.

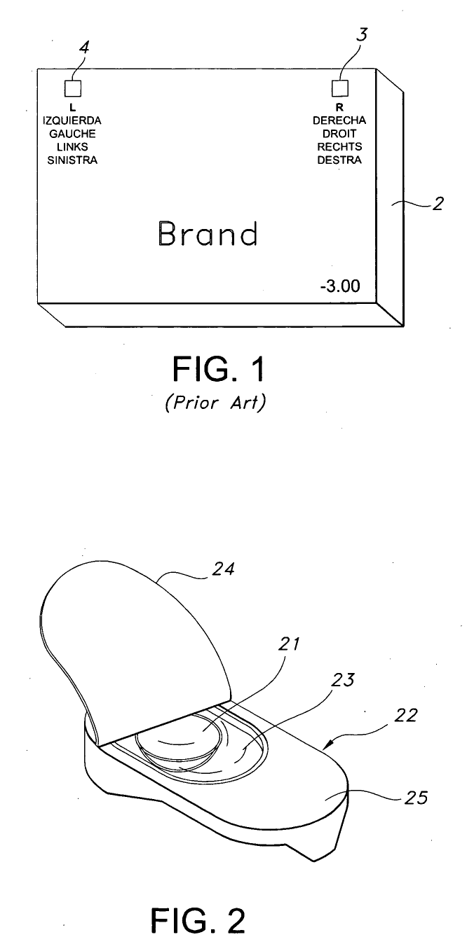

### DETAILED DESCRIPTION OF VARIOUS PREFERRED EMBODIMENTS

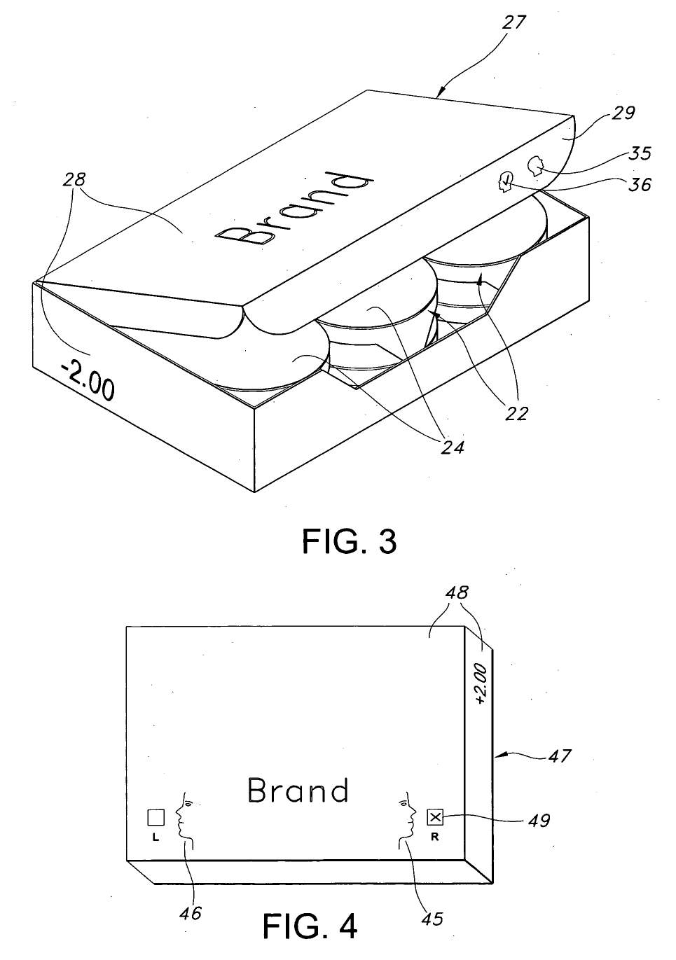

Referring to FIGS. 2 and 3, a conventional manner of packaging a contact lens 21 is in a blister package 22. The blister package may have various shapes, but in general, the package 22 will include a compartment 23 for holding an individual contact lens 21 immersed in an aqueous solution. In the illustrated embodiment, compartment 23 has the form of an indentation in base 25. Cover sheet 24 is initially sealed to base 25 (as seen in FIG. 3), so as to sealingly enclose the contact lens 21 in compartment 23. As seen in FIG. 2, the cover sheet 24 can be peeled back, or removed, in order to access the contact lens 21.

FIG. 3 illustrates multiple packages 22 contained in secondary carton 27. In the illustrated embodiment, six packages 22 are enclosed in carton 27, three on a bottom layer and three on a top layer. Conventionally, all packages 22 in carton 27 are lenses of the same prescription. Carton 27 may include various graphics 28, such as textual graphics representing brand and prescription. Additionally, on flap 29 of carton 27, there are non-textual graphics of a right-side facial profile 35 and a left-side facial profile 36. More specifically, right-side facial profile 35 is a full silhouette of the right side of a human head, and left-side facial profile 36 is a full silhouette of the left side of a human head, with these two silhouettes separated from one another. In this illustrated embodiment, both silhouettes have a blank box, in order to permit the prescribing practitioner, or the contact lens wearer, to place a mark in the box so as to designate either right or left eye placement of the lenses contained in the carton. In the illustrated embodiment, a checkmark has been placed in left facial profile 36, thereby designating the lenses as intended for placement in the left eye, i.e., the lenses contained in carton 27 correspond to the contact lens wearer's left eye prescription. Different marks may be made, for example, by circling the left facial profile 36.

FIG. 4 illustrates another embodiment of this invention. Secondary carton 47 again encloses multiple contact lens packages, and includes graphics 48, such as graphics indicating brand and prescription of the lenses contained in the carton 47. In this embodiment, right-side facial profile 45 is a partial silhouette of the right side of a human head, and left-side facial profile 46 is a partial silhouette of the left side of a human head. In this illustrated embodiment, box 49, adjacent to right-side facial profile 45, has been marked to designate the lenses contained in the carton for placement on the right eye. Optionally, graphics of “R” and “L” may be included, as shown in FIG. 4, but it will be appreciated that these “R” and “L” graphics are unnecessary, in that the right-side and left-side facial profiles 41, 42 fully convey either right or left eye placement, respectively, without the need for the R and L alphanumeric characters.

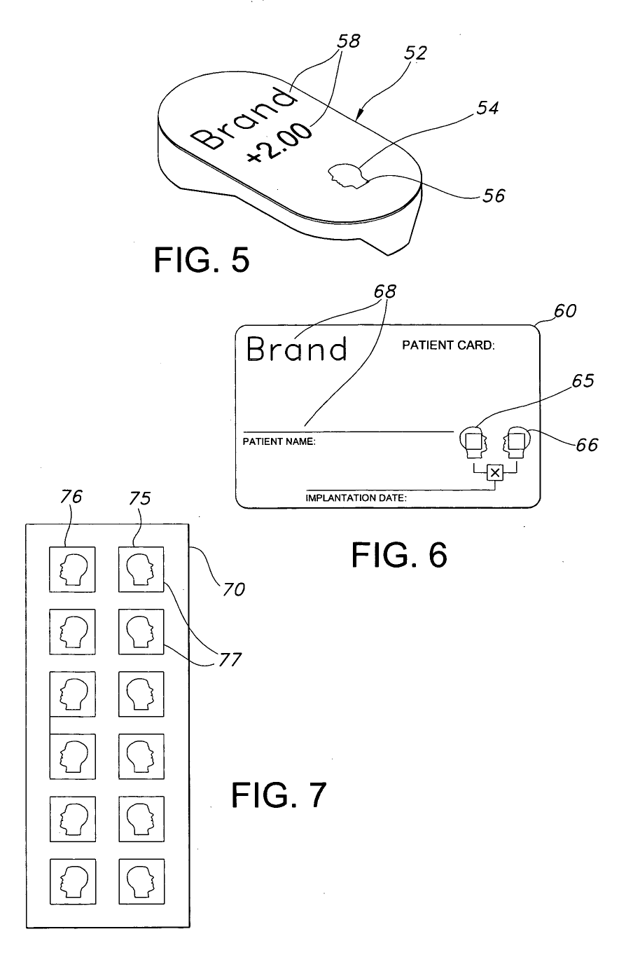

FIG. 5 illustrates another embodiment of this invention. Blister package 52 encloses a contact lens beneath cover sheet 54. Package 52 may include graphics 58, including information such as brand and prescription. Additionally, package 52 includes graphics on the cover sheet 54 of a left-side facial profile 56 but no graphics of a right-side facial profile. For example, at the time an order for lenses is placed with the manufacturer or other supplier, the order can indicate, in addition to brand, prescription and quantity, whether the order is intended for the patient's right or left eye. The manufacturer or other supplier can then apply to the blister package either the right- or left-side facial profile. As an example, as shown in FIG. 5, the indicated left-side facial profile 56 may be printed on the cover sheet 54. Alternately, this single facial profile could instead be applied to the secondary carton for the blister packages, or to both the blister packages and their carton.

FIG. 6 illustrates card 60 to be enclosed in a carton containing an ophthalmic lens. In other words, card 60 is an insert to be packaged along with an ophthalmic lens. In the illustrated embodiment, card 60 is printed with graphics 68, including the right-side facial profile 65 and the left-side facial profile 66 separated from one another. As an example, card 60 may be packaged with an intraocular lens. After implanting the lens, the doctor can easily indicate on the card whether the lens was implanted in the right or left eye, by marking either the right-side facial profile or the left-side facial profile. The doctor can add any desired supplemental information to the card, such as patient name and implantation date, and retain the card in the patient's records.

FIG. 7 illustrates another package insert 70. Insert 70 may be enclosed in a secondary carton along with multiple contact lens blister packages; for example, insert 70 may be enclosed in carton 27 of FIG. 3 or carton 47 of FIG. 4. Insert 70 includes a column of right-side facial profiles 75 and a column of left-side facial profiles 76. Each individual right- and left-side profile is printed on an individual adhesive-backed strip of paper 77 which can be peeled from insert 70. As an example, insert 70 may be inserted in carton 27 of FIG. 3, the lenses of which have been prescribed for the wearer's left eye. The contact lens wearer can remove from insert 70 one or more individual strips 77 having the left-side profile 76, and re-adhere the individual strips to blister packages 22. Thus, if the contact lens wearer wants to carry extra blister packages but not the entire carton, the wearer can designate the lens in the package removed from carton 27 for left eye placement.

It will be appreciated that the this invention, wherein either a right-side facial profile, a left-side profile, or both are applied to lens packaging material, permit simple identification and designation of right or left placement of a lens. The non-textual right- and left-side facial profiles fully convey right and left eye placement, respectively, without the need for any alphanumeric graphics relating to eye placement. Additionally, the packaging materials may be distributed to multiple geographic areas, including non-English speaking countries and regions not using the Roman alphabet, without the need for any textual description of right and left eye placement or translations thereof.

The illustrated embodiments demonstrate various aspects of the present invention. However, it is to be understood that these embodiments are for illustrative purposes only and do not purport to be wholly definitive as to the conditions and scope of the invention. While the invention has been described in connection with various preferred embodiments, numerous variations will be apparent to a person of ordinary skill in the art given the present description, without departing from the spirit of the invention and the scope of the appended claims.

I claim:

1. Packaging material for ophthalmic lenses including graphics comprising at least one of a right-side facial profile or a left-side facial profile.

2. The packaging material of claim 1, wherein the graphics include both the right-side and left-side facial profiles that are spatially separated from one another on the packaging material.

3. The packaging material of claim 1, wherein the right-side facial profile includes a silhouette of the right side of a human head, or the left-side facial profile includes a silhouette of the left side of a human head.

4. The packaging material of claim 1, wherein the graphics include a silhouette of the right side of a human head, and a silhouette of the left side of a human head.

5. The packaging material of claim 1, wherein the graphics are included on a secondary carton, the secondary carton enclosing primary packages containing ophthalmic lenses.

6. The packaging material of claim 5, wherein the primary packages contain contact lenses.

7. The packaging material of claim 5, wherein the right-side facial profile includes a silhouette of the right side of a human head, or the left-side facial profile includes a silhouette of the left side of a human head.

8. The packaging material of claim 5, wherein the graphics include a silhouette of the right side of a human head, and a silhouette of the left side of a human head.

9. The packaging material of claim 1, wherein the graphics are included on a blister package containing a contact lens.

10. The packaging material of claim 9, wherein the blister package includes a cover sheet sealingly enclosing a contact lens compartment, and the graphics are included on the cover sheet.

11. The packaging material of claim 9, wherein the right-side facial profile includes a silhouette of the right side of a human head, or the left-side facial profile includes a silhouette of the left side of a human head.

12. The packaging material of claim 9, wherein the graphics include a silhouette of the right side of a human head, and a silhouette of the left side of a human head.

13. The packaging material of claim 1, wherein the graphics are included on an insert enclosed in a carton containing an ophthalmic lens.

14. The packaging material of claim 13, wherein the ophthalmic lens is contained in a primary package, and the primary package and the insert are enclosed in a secondary carton.

15. The packaging material of claim 13, wherein the right-side facial profile includes a silhouette of the right side of a human head, or the left-side facial profile includes a silhouette of the left side of a human head.

16. The packaging material of claim 13, wherein the graphics include a silhouette of the right side of a human head, and a silhouette of the left side of a human head.

17. A method for identifying an ophthalmic lens for placement in or on the right or left eye, comprising, designating the right or left eye placement on packaging material for the lens with, respectively, a right-side facial profile or a left-side facial profile.

18. The method of claim 17, wherein: when the lens is intended for placement in or on the right eye, the packaging material includes graphics including the right-side facial profile and not the left-side facial profile; and when the lens is intended for placement in or on the left eye, the packaging material includes graphics including the left-side facial profile and not the right-side facial profile.

19. The method of claim 17, wherein packaging material for the ophthalmic lens includes graphics comprising both a right-side facial profile and a left-side facial profile that are spatially separated from one another on the packaging material, and right or left eye placement is designating by marking one of the right or left facial profiles.

20. A method for distinguishing placement of an ophthalmic lens for the right eye or the left eye, comprising, including on packaging material for the lens a right-side facial profile and a left-side facial profile that are spatially separated from one another.
